(19) United States
(12) Patent Application Publication
(10) Pub. No.: US 2017/0252425 A1
(43) Pub. Date: Sep. 7, 2017

(54) ZIKA VIRUS VIRUS LIKE PARTICLE

(71) Applicant: VLP Therapeutics, LLC, Wilmington, DE (US)

(72) Inventors: Wataru AKAHATA, Kensington, MD (US); Ryuji UENO, Easton, MD (US)

(73) Assignee: VLP Therapeutics, LLC, Wilmington, DE (US)

(21) Appl. No.: 15/448,901

(22) Filed: Mar. 3, 2017

Related U.S. Application Data
(60) Provisional application No. 62/303,571, filed on Mar. 4, 2016.

(51) Int. Cl. A61K 39/12; G01N 33/569; C07K 14/005; C07K 16/10; C12N 7/00

(52) U.S. Cl. CPC A61K 39/12; C07K 16/10; C12N 7/00; C07K 14/005; G01N 33/56983; A61K 2039/53

(57) ABSTRACT

Provided is a virus like particle comprising one or more zika virus structural proteins, and a composition or vaccine comprising thereof, its use in the prevention or treatment of zika virus disease. The zika virus structural protein contains at least one amino acid alteration in the envelope protein.

### CROSS REFERENCE TO RELATED APPLICATIONS

This application claims the benefit of U.S. provisional Patent Application No. 62/303,571 filed on Mar. 4, 2016, the entire contents of which are incorporated by reference herein.

### TECHNICAL FIELD

The present application relates to a virus like particle comprising one or more zika virus structural proteins, and a composition or vaccine comprising thereof, its use in medicine, particularly in the prevention or treatment of zika virus disease.

### BACKGROUND ART

Zika virus is an emerging mosquito-borne virus that was first identified in Uganda in 1947 in rhesus monkeys through a monitoring network of sylvatic yellow fever. It was subsequently identified in humans in 1952 in Uganda and the United Republic of Tanzania. Outbreaks of Zika virus disease have been recorded in Africa, the Americas, Asia and the Pacific.

During large outbreaks in French Polynesia and Brazil in 2013 and 2015 respectively, national health authorities reported potential neurological and auto-immune complications of Zika virus disease. Recently in Brazil, local health authorities have observed an increase in Guillain-Barré syndrome which coincided with Zika virus infections in the general public, as well as an increase in babies born with microcephaly in northeast Brazil. Agencies investigating the Zika outbreaks are finding an increasing body of evidence about the link between Zika virus and microcephaly. However, more investigation is needed to better understand the relationship between microcephaly in babies and the Zika virus. Other potential causes are also being investigated.

Zika virus is transmitted to people through the bite of an infected mosquito from the Aedes genus, mainly Aedes aegypti in tropical regions. This is the same mosquito that transmits dengue, chikungunya and yellow fever. However, sexual transmission of Zika virus has been described in 2 cases, and the presence of the Zika virus in semen in 1 additional case.

Zika virus disease outbreaks were reported for the first time from the Pacific in 2007 and 2013 (Yap and French Polynesia, respectively), and in 2015 from the Americas (Brazil and Colombia) and Africa (Cape Verde). In addition, more than 13 countries in the Americas have reported sporadic Zika virus infections indicating rapid geographic expansion of Zika virus.

According to the Zika virus fact sheet updated on Sep. 6, 2016 provided by WHO and available on the web, there is no specific treatment or vaccine currently available. (http://www.who.int/mediacentre/factsheets/zika/en/)

Virus-like particles (VLPs) are multiprotein structures that mimic the organization and conformation of authentic native viruses but non-infectious because they do not contain any viral genome, potentially yielding safer vaccine candidates. A handful of prophylactic VLP-based vaccines is currently commercialized worldwide: GlaxoSmithKline's Engerix® (hepatitis B virus) and Cervarix® (human papillomavirus), and Merck and Co., Inc.'s Recombivax HB® (hepatitis B virus) and Gardasil® (human papillomavirus) are some examples. Other VLP-based vaccine candidates are in clinical trials or undergoing preclinical evaluation, such as, influenza virus, parvovirus, Norwalk and various chimeric VLPs. Many others are still restricted to small-scale fundamental research, despite their success in preclinical tests. The implications of large-scale VLP production are discussed in the context of process control, monitorization and optimization. The main up- and down-stream technical challenges are identified and discussed accordingly. Successful VLP-based vaccine blockbusters are briefly presented concomitantly with the latest results from clinical trials and the recent developments in chimeric VLP-based technology for either therapeutic or prophylactic vaccination.

Up to now, VLP-based vaccines have been produced for more than 30 different viruses that infect human and other animals. The examples include AAV (Adeno-associated virus), H5N3 (Avian influenza), BFDV (Budgerigar fledgling disease virus), BTV (Bluetongue virus), Ebola, Enterovirus 71, GHPV (Goose hemorrhagic polyoma virus), HBV (Hepatitis B virus), HCV (Hepatitis C virus), HDV (Hepatitis δ virus), HEV (Hepatitis E virus), HIV, HPV (Human papillomavirus), IBDV (Infectious bursal disease virus), Influenza A, Influenza A H1N1, Influenza A H3N2, JC polymavirus, Margurg, MS2, IPCV (Indian peanut clump virus), NDV (Newcastle disease virus), No (Norovirus), Nv (Norwalk virus), PhMV (Physalis mottle virus), Polymavirus, PPV (Porcine parvovirus), RHDV (Rabbit hemorrhagic disease virus), Rotavirus, SARS, SIV (Simian immunodeficiency virus), SV40 (Simian virus 40), SVDV (Swine vesicular disease virus) and so on. (Expert Rev. Vaccines 9(10), 1149-1176, 2010).

### SUMMARY OF THE INVENTION

In a first aspect, the present application provides a virus like particle comprising one or more zika virus structural proteins including the envelope protein, wherein the envelope protein of the zika virus structural protein contains at least one alteration in the amino acid sequence.

In a second aspect, the present application provides a nucleic acid molecule comprising or consisting of a nucleotide sequence that encodes the zika virus structural protein contained in the virus like particle provided in the first aspect of the present application.

In a third aspect, the present application provides a composition or vaccine comprising the virus like particle provided in the first aspect and/or the nucleic acid molecule provided in the second aspect.

In a fourth aspect, the present application provides a method of producing an antibody or anti-serum comprising a neutralizing antibody, comprising contacting the virus like particle provided in the first aspect of the present application and/or the nucleic acid molecule provided in the second aspect of the present application to a mammal.

In a fifth aspect, the present application provides a method of treating or preventing zika virus infection/disease or a method of inducing and/or enhancing immune response against a zika virus in a mammal, comprising administering the composition provided in the third aspect of the present application to the mammal.

In a sixth aspect, the present application provides a method of producing the virus like particle provided in the first aspect of the present application, comprising: culturing a cell which is transfected with a gene encoding the at least one zika virus structural proteins contained in the virus like particle; and recovering virus like particle from the cell culture. This aspect may further comprise the step of preparing a gene comprising a nucleotide sequence encoding the at least one zika virus structural proteins contained in the virus like particle.

In a seventh aspect, the present application provides a virus like particle for use in a method or kit of diagnosing zika virus infection in a mammal.

### BRIEF DESCRIPTION OF DRAWINGS

FIG. 1 shows representative constructs of ZIKV VLPs.

FIG. 2 shows envelope proteins in the supernatants of the 293F cells transfected with the ZIKV VLP constructs shown in FIG. 1.

### DESCRIPTION OF EMBODIMENTS

### (1) A Virus Like Particle Comprising One or More Zika Virus Structural Proteins Including the Envelope Protein

In the first aspect, the present application provides a virus like particle comprising one or more zika virus structural proteins including the envelope protein, wherein at least one amino acid in the envelope protein is altered from its naturally occurred structure.

Zika virus (ZIKV), has a positive-sense, single-stranded RNA genome approximately 11 kilobases in length. The genome contains 5′ and 3′ untranslated regions flanking a single open reading frame (ORF) that encodes a polyprotein that is cleaved into three structural proteins: the capsid (C), premembrane/membrane (prM), and envelope (E), and eight non-structural proteins (NS1, NS2A, NS2B, NS3, NS4A, 2K, NS4B, and NS5). A previous genetic study using nucleotide sequences derived from the NS5 gene indicated three ZIKV lineages: East African (one strain examined), West African (three strains examined), and Asian (one strain examined) (Haddow A D, Schuh A J, Yasuda C Y, Kasper M R, Heang V, et al. (2012) Genetic Characterization of Zika Virus Strains: Geographic Expansion of the Asian Lineage. PLoS Negl Trop Dis 6(2): e1477). Further, ZIKV maximum likelihood phylogenetic tree has been proposed (Rafael A. Larocca, Peter Abbink, Jean Pierre S. Peron, Paolo M. de A. Zanotto, M. Justin Iampietro, Alexander Badamchi-Zadeh, Michael Boyd, David Ng'ang'a, Marinela Kirilova, Ramya Nityanandam, Noe B. Mercado, Zhenfeng Li, Edward T. Moseley, Christine A. Bricault, Erica N. Borducchi, Patricia B. Giglio, David Jetton, George Neubauer, Joseph P. Nkolola, Lori F. Maxfield, Rafael A. De La Barrera, Richard G. Jarman, Kenneth H. Eckels, Nelson L. Michael, Stephen J. Thomas & Dan H. Barouch (2016) Nature 536, 474-478).

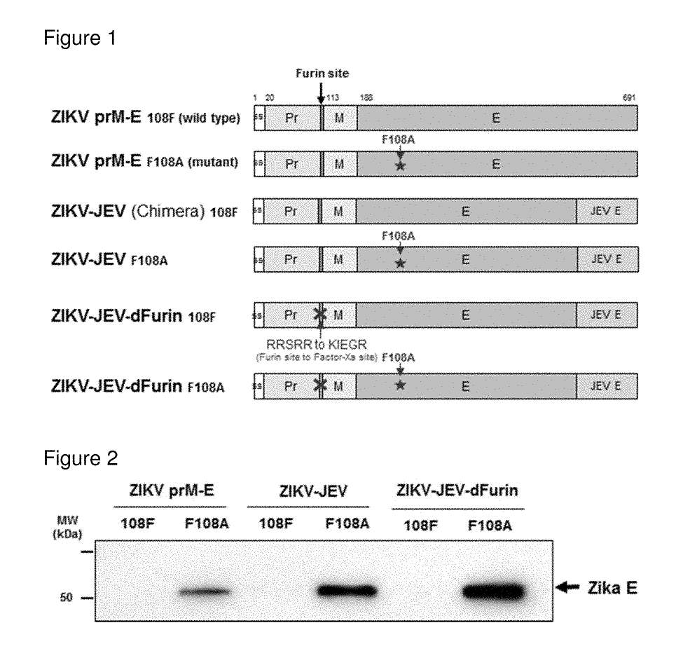

A list of ZIKV strains is shown, for example, in Nature 536, 474-478 (2016) in the extended data FIG. 1. ZIKV virus structural proteins have been identified and available at various public databases such as GenBank database. Representative zika virus strains may include Strain MR766 Accession No. NC012532/YP002790881 and Strain 21106033 Accession No. KU312312/ALX35659. Their envelope proteins have the amino acid sequences of SEQ ID Nos: 11 and 12, respectively. According to this application, the at least one alteration in the amino acid sequence may be between amino acid position 90 and amino acid position 121, especially between amino acid position 99 and amino acid position 110, particularly at 108 of SEQ ID Nos: 11 or 12. For example, Phe at position 108 of SEQ ID Nos: 11 or 12 may be altered to Ala.

According to this application, the envelope protein of a zika virus may be obtained from a database and aligned with SEQ ID Nos: 11 and 12 to identify a position corresponding to 90-121aa, preferably 99-110aa such as 108aa of SEQ ID Nos: 11 or 12. Examples of zika virus may include Brazil—ZKV2015 (GenBank: KU497555/AMD16557), zika virus MR 766 GenBank: DQ859059/ABI54475,) Zika virus strain PRVABC59 (GenBank: KU501215/AMC13911) and zika virus strain BeH815774 (Genebank: KU365780/AMA12087). Amino acid sequences of the envelope region of those strains are available from the GenBank database.

A part of the C terminus region of zika virus envelope protein may be replaced with corresponding region of an envelope protein of a flavivirus. Examples of flavivirus may include Dengue virus and Japanese Encephalitis virus. The C-terminus 80-250 amino acids region of the zika virus envelope protein may be replaced with the corresponding C-terminus amino acid region of a flavivirus envelope protein to give a chimeric envelope protein. For example, the C-terminus 202 amino acids of zika envelope protein of SEQ ID No: 11 or SEQ ID NO: 12 or any zika envelope protein corresponding to the C-terminus 202 amino acids of SEQ ID NO: 11 or SEQ ID NO: 12 may be replaced with the C-terminus 199 amino acids of Dengue typel virus (WestPac strain, GenBank Accsession No: U88535) envelope protein of SEQ ID NO: 13, or any Dengue virus envelope protein corresponding to the C-terminus 199 amino acids of SEQ ID NO: 13. Alternatively, the C-terminus 101 amino acids of zika virus envelope protein of SEQ ID No: 11 or 12 or any ZIKV E region corresponding to the C-terminus 101 amino acids of SEQ ID NO: 11 or 12 may be replaced with the C-terminus 101 amino acids of Japanese Encephalitis virus (JEV) envelope protein, for example that of nakayama strain, Genebank# EF571853 of SEQ ID NO: 14, or a JEV envelope protein C-terminus region corresponding to the C-terminus 101 amino acids of SEQ ID NO: 14.

In this embodiment, the zika virus structural protein comprise envelope protein and preferably, comprise a prM protein and an envelope protein. The furin protease recognition sequence of Arg-Arg-Ser-Arg-Arg (SEQ ID NO: 19) in the zika virus prM protein may be disrupted by altering the sequence to Factor Xa (FXa) recognition sequence of Lys-Ile-Glu-Gly-Arg (SEQ ID NO: 20).

When the zika virus structural protein comprises prM and envelope proteins, all of the pr region, M region and envelope region may be derived any of ZIKV strains, for example, strains shown in Nature 536, 474-478 (2016). All of the pr, M and envelope regions may be derived from a single ZIKV strain. Alternatively, each of the regions may be derived from distinct ZIKV strains.

A construct for preparing the zika virus structural proteins may further comprise an amino acid sequence corresponding to the initiation codon and a signal sequence to the amino terminal of the zika virus structural protein. For example, the construct may comprise an initiation codon M and a signal sequence that are followed by prM and envelope proteins. The signal sequence may be derived any of ZIKV strains as discussed above.

The region comprising at least one amino acid alternation may be preferably between amino acid position 188 and the C-terminal, more preferably between amino acid position 277 and amino acid position 308, especially between amino acid position 286 and amino acid position 297, particularly 295 of a protein of SEQ ID Nos: 1, 3, 5, 7, 15 or 17 which comprises initiation codon M and signal peptide of SEQ ID No: 9 or 10 followed by prM and envelope region of zika Virus (MR766 strain, GenBank Accsession No: NC012532), (Z1106033 strain, GenBank Accsession No: KU312312), or chimeric envelope region. The at least one amino acid alternation may be between the positions determined as the above-identified positions when the amino acid sequence of a protein comprising initiation codon M, signal peptide followed by prM and envelope region of a zika virus, a fragment thereof or a chimeric envelope region is aligned with the amino acid sequence represented by SEQ ID Nos: 1, 3, 5, 7, 15 or 17.

As used herein, “a position corresponding to” or recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm. By aligning the sequences, one skilled in the art can identify corresponding residues.

With the exception of comprising at least one amino acid alteration in the envelope protein, a zika virus structural protein contained in the virus like particle may be a naturally occurring viral structural protein or a modified protein thereof. In one embodiment, the modified protein has at least 70%, 75%, 80%, 85%, 90%, 95% or 98% amino acid sequence identity to a naturally occurring viral structural protein including prM and envelope protein. When the pr, M and envelope regions are derived from different ZIKV strains, the modified protein has at least 70%, 75%, 80%, 85%, 90%, 95% or 98% amino acid sequence identity to the combination of naturally occurring pr, M and envelope regions from which the modified protein have been derived. In one embodiment, the modified protein is a mutant where at most 10% of the amino acids are deleted, substituted, and/or added to a naturally occurring viral structural protein including the pr, M and envelope regions. The sequence identity may be determined by conventional methods.

In another embodiment, when the zika virus envelope protein is a chimeric protein with a part of flavivirus envelope protein such as Dengue type 1 virus or Japanese encephalitis virus envelope protein, the modified zika virus structural protein has at least 70%, 75%, 80%, 85%, 90%, 95% or 98% amino acid sequence identity to a naturally occurring prM and the chimeric envelope proteins. In one embodiment, the modified protein is a mutant where at most 10% of the amino acids are deleted, substituted, and/or added to a naturally occurring prM and chimeric envelope proteins.

According to the present application, one or more zika virus structural proteins or fragments thereof discussed above may be used as long as they spontaneously assemble into a particulate structure. For example, when eukaryotic cells expressing a gene encoding prM and envelope proteins of zika virus are cultured, the proteins are generated by the cells and assemble to give VLPs, and the VLPs can be collected from the cell culture supernatant.

The present application addresses one or more of the above needs by providing VLPs, vectors encoding the VLPs, and antibodies (and antibody-like molecules including aptamers and peptides) that specifically bind to the antigen, together with the uses thereof (either alone or in combination) in the prevention or treatment of zika virus infections.

As used in the specification and claims, the term “antibody” refers to a molecule which is capable of binding to an epitope or antigenic determinant. The term covers a whole antibody and an antigen-binding fragment thereof, including a single-chain antibody. Such antibodies may include human antigen binding antibody fragments and include, but are not limited to, Fab, Fab′ and F(ab′)2, Fd, single-chain Fvs (scFv), single-chain antibodies, disulfide-linked Fvs (sdFv) and fragments comprising either a VL or VH domain. The antibodies can be from any animal origin including birds and mammals. Preferably, the antibodies are mammalian e.g. human, murine, rabbit, goat, guinea pig, camel, horse and the like, or other suitable animals e.g. chicken. As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described, for example, in U.S. Pat. No. 5,939,598, the disclosure of which is incorporated herein by reference in its entirety.

Except for having at least one alteration in the envelope protein, a zika virus structural protein may be a naturally occurring protein or modified protein of the naturally occurring protein or a fragment of the naturally occurring protein or the modified protein. The modified protein may be a fragment of the naturally occurring virus structural protein.

In one embodiment, the modified protein derived from a zika virus structural protein has at least 70%, 75%, 80%, 85%, 90%, 95% or 98% amino acid sequence identity to the naturally occurring protein. In one embodiment, the modified protein derived from a zika virus is a mutant where at most 10% of the amino acids are deleted, substituted, and/or added based on the naturally occurring viral structural protein derived from the zika virus.

In one embodiment, the present application provides a virus like particle obtained by using a construct comprising a zika virus structural protein having an amino acid sequence represented by any one of SEQ ID Nos: 1, 3, 5, 7, 15 and 17.

Each of those sequences represents a protein containing the following regions: initiation codon: M (laa), signal sequence (2-19aa), pr sequence (20-112aa), M sequence (113-187aa), and Envelope region (188aa-C-terminal). In one embodiment, a virus like particle comprising a viral structural protein having the prM and envelope regions of SEQ ID Nos: 1, 3, 5, 7, 15 and 17, i.e. comprising amino acid sequences of from position 20 to the C-terminal of those proteins may be provided.

The modified zika virus structural protein may have at least 70%, 75%, 80%, 85%, 90%, 95% or 98% amino acid sequence identity to an amino acid sequence represented by any one of SEQ ID Nos: 1, 3, 5, 7, 15 and 17, or an amino acid sequence from position 20 to the C-terminal of any one of those sequences. Also, the modified zika virus structural protein may be a mutant where at most 10% of the amino acids are deleted, substituted, and/or added based on the zika virus structural protein having an amino acid sequence represented by any one of SEQ ID Nos: 1, 3, 5, 7, 15 and 17, or an amino acid sequence from position 20 to C-terminal of any one of those sequences.

### (2) Nucleotide and Vector

In the second aspect, the present application provides a nucleic acid molecule comprising or consisting of a nucleotide sequence encoding the construct for preparing the virus like particle as provided in the first aspect of the present invention.

In one embodiment, the present application provides a nucleic acid molecule comprising a nucleotide sequence that encodes a zika virus structural protein that provide the virus like particle as described above or a construct for preparing the zika virus structural protein.

In one embodiment, the present application provides an expression vector comprising the nucleic acid molecule as described above, wherein the vector optionally comprises an expression control sequence operably linked to the nucleic acid molecule.

Examples of expression control sequences include, but are not limited to, promoter such as CMV promoter, phage lambda PL promoter, the E. coli lac, phoA and tac promoters, the SV40 early and late promoters, and promoters of retroviral LTRs.

In one embodiment, the present application provides an expression vector for a zika virus structural protein, which comprises a construct consisting of a nucleotide sequence represented by any one of SEQ ID Nos: 2, 4, 6, 8, 16 and 18.

In one embodiment, the present application provides a nucleic acid molecule which is modified from the nucleic acid molecule having a nucleotide sequence represented by any one of SEQ ID Nos: 2, 4, 6, 8, 16 and 18. The modified nucleic acid molecule may have at least 70%, 75%, 80%, 85%, 90%, 95% or 98% nucleotide sequence identity to the nucleic acid molecule having a nucleotide sequence represented by any one of SEQ ID Nos: 2, 4, 6, 8, 16 and 18. Also, the modified nucleic acid molecule may be a mutant where at most 10% of the amino acids are deleted, substituted, and/or added based on the nucleic acid molecule having a nucleotide sequence represented by any one of SEQ ID Nos: 2, 4, 6, 8, 16 and 18.

### (3) Composition or Vaccine

In the third aspect, the present application provides a composition or vaccine comprising the virus like particle provided in the first aspect of the present application and/or the nucleic acid molecule provided in the second aspect of the present invention.

In one embodiment, the present application provides a composition comprising the zika virus virus like particle as described above or the nucleic acid molecule as described above.

The composition may further comprise a pharmaceutically acceptable carrier and/or adjuvant. Examples of adjuvant include, but are not limited to Ribi solution (Sigma Adjuvant system, Sigma-Aldrich).

The pharmaceutical composition of the present application may contain a single active ingredient or a combination of two or more active ingredients, as far as they are not contrary to the objects of the present invention. For example, cytokines including chemokines, antibodies against a cytokine such as anti-TNF antibody (e.g. infliximab, adalimumab), anti-VEGF antibody (e.g. bevacizumab and ranibizumab), cytokine receptor antagonist such as anti HER2 antibody (e.g. Trastuzumab), anti EGF receptor antibody (e.g. Cetuximab), anti VEGF aptamer (e.g. Pegaptanib) and immunomodulator such as cyclosporine, tacrolimus and ubenimex may be used for the combination therapy.

In a combination of plural active ingredients, contents of the respective ingredients may be suitably increased or decreased in consideration of their therapeutic effects and safety.

The term “combination” used herein means two or more active ingredients are administered to a patient simultaneously in the form of a single entity or dosage, or those active ingredients are administered to a patient as separate entities either simultaneously or sequentially with no specific time limits, wherein such administration provides therapeutically effective levels of the two components in the body, preferably at the same time.

In one embodiment, the composition is a vaccine composition including a DNA vaccine. In one embodiment, the DNA vaccine provided by the present invention comprises CpG containing oligonucleotide.

### (4) Method of Producing an Antibody

In the fourth aspect, the present application provides a method of producing an antibody against a zika virus or an antiserum containing a neutralizing antibody against a zika virus, comprising contacting the virus like particle provided in the first aspect of the present application and/or the nucleic acid molecule provided in the second aspect of the present invention to a mammal.

The antibody produced in this aspect may be used for passive immunization against a zika virus-causing pathogen in a mammal by administering the same to the mammal, and thus prevent the mammal from zika virus infection or treat a disease or condition caused by zika virus infection in the mammal.

The antibody produced in the fourth aspect of the present application may be humanized using a conventional technique. Thus, in one embodiment, the method provided in the fourth aspect of the invention further comprises a step of humanizing a non-human mammal produced antibody. The antibody or humanized antibody provided by this aspect may be used for preventing a human subject from zika virus infection or for treating a disease or condition caused by zika virus infection in the subject.

The antibody produced according to this aspect may be used in vitro to select a subpopulation from immune cells such as B-cell and T-cell derived from the patient, which are then re-administered to the patient.

Antiserum can be obtained by the conventional manner. Blood samples are taken from the immunized non-human animal, and the blood is processed so as to obtain the antiserum, i.e. the antibody-containing liquid component of the blood. The non-human mammal is preferably selected from the group consisting of rat, mouse, hamster, pig, rabbit, horse, donkey, goat, sheep, guinea pig, lama, and non-human primate (e.g. chimp).

(5) Method of Treating a Disease Caused by a Zika Virus Infection in a Subject or Preventing a Subject from Zika Virus Infection

In the fifth aspect, a method of treating a disease or condition caused by zika virus infection such as zika fever in a subject, wherein the virus like particle provided in the first aspect, the nucleotide molecule provided in the second aspect, or the composition provided in the third aspect is administered to the subject. By administering the above listed VLP, nucleotide molecule or composition to the subject, immune response against a zika virus can be enhanced and thus, the disease or condition caused by the zika virus infection can effectively be treated. In this aspect, the VLP, nucleotide molecule or composition may be administered to the patient locally to the affected organ or systemically.

A method of preventing a subject from a zika virus infection or from a disease caused by a zika virus infection, comprising administering the virus like particle of the first aspect, the nucleotide molecule provided in the second aspect or the composition of the third aspect to the subject. The disease caused by a zika virus may be zika fever.

According to the present application, the virus like particle can also be applied for immune therapy. The VLP may be applied ex vivo to cells derived from the patient or a human cell line which are subsequently administered to the patient.

### (6) Method of Producing the Virus Like Particle

In the sixth aspect, the present application provides a method of producing the virus like particle provided in the first aspect of the present invention, comprising culturing a cell which is expressing a gene coding for the viral structural protein; and recovering the virus like particle from the cell culture.

Various host-vector systems may be used for expression of the virus like particle. Eukaryotic cells can be used for the method provided by the sixth aspect of the present application. Examples of eukaryotic cells include, but are not limited to, insect cells (e.g. sf9 cells, H5 cells), yeast cells (e.g. S. cerevisiae) and mammalian cells (e.g. CHO cells, human embryonic kidney (HEK) 293F cells). Bacteria (e.g. E. coli) may also be used for the method provided by the sixth aspect of the present application. Vector used for the method provided by the second aspect of the present application comprises a nucleic acid molecule encoding the virus like particle to be expressed. Cells may be transfected with the vector using conventional methods (e.g. lipofection, electroporation). A skilled person can select culture medium or with DNA methyl transferase inhibitors and histone deacetylase inhibitors such as sodium butyrate, depending on cells employed. After the transfection, virus like particle can be produced in the cells and/or culture supernatant. Virus like particle may be recovered from the culture supernatant and purified using ultracentrifugation.

The VLPs described as above may be prepared by stable cell line. The stable cell line can be prepared by using the above-described vectors and according to conventional procedures. For example, the following procedures may be employed to generate a stable cell line:

1. Transfect cells such as 293F cells with a VLP expression plasmid containing a selection marker such as hygromycin B.

2. Incubate the transfected cells for one day

3. Culture the transfected cells in a selection medium containing such as Hygromycin at 150-200 ug/ml for 1-2 weeks.

4. Choose the cells that can grow and be split at least once in the selection medium.

5. Isolate a single cell and confirm the expression of the VLP in the supernatants by western blotting.

The virus like particles of the present application do not replicate and therefore, have highly safe profiles.

### (7) Kit or Method of Diagnosing Zika Virus Infection

In the seventh aspect, the present application provides a virus like particle for use in a method or kit of diagnosing zika virus infection in a mammal. By using the virus like particles, an enzyme-linked immune-sorbent assay (ELISA) diagnostic kit capable of detecting an antibody specific to the zika virus could be produced.

The present application will be described in detail with reference to the following example, which, however, is not intended to limit the scope of the present invention.

### Example 1

### Preparation of Zika Virus Virus Like Particle

Zika virus viral structural proteins introduced at least one alteration in the envelope protein were used to form a zika virus virus like particle. Namely, amino acid Phe at position 108 of Zika virus envelope protein of Strain MR766 and Strain 21106033 (SEQ ID Nos: 11 and 12) was altered to Ala (F108A). In addition, chimera envelope proteins wherein C-terminal region of the zika virus envelope protein (299aa-500aa of SEQ ID NO: 11 and 303-504aa of SEQ ID No: 12) was replaced to the corresponding region (SEQ ID NO: 13) of Dengue virus type 1 (WestPac strain, GenBank Accsession No: U88535) envelope protein were also used. Amino acid Phe at position 108 of the chimera envelope proteins was also altered to Ala (F108A).

Constructs for preparing zika virus virus like protein comprising initiation codon M, signal peptide, prM region and the modified envelope region were prepared (SEQ ID Nos: 1, 3, 5 and 7). In this example, envelope region corresponds to 188aa-C-terminal of SEQ ID Nos: 1, 3, 5 and 7. In the examples, mutation in the envelope region is expressed using the amino acid number starting from the envelope region.

To construct wild type zika virus virus like particle expression vector, DNA encoding structure proteins of Zika virus (ZIKV, Genbank# NC_012532, YP_002790881 and Genebank# KU312312) were generated by gene synthesis (GeneArt, Thermo Fisher Scientific). The signal sequence (ss), pre-Membrane (prM), and Envelope (E) genes were cloned into pUC119 based expression vector with cytomegalovirus promoter and poly A signals. ZIKV VLPs of this example were prepared by using genes encoding the modified or modified chimeric envelope region instead of the wild type envelope gene. The nucleotide sequences of the constructs are shown in SEQ ID Nos: 2, 4, 6 and 8. To prepare VLPs, expression vectors comprising the constructs were transfected to 293F cells using GeneX plus transfection reagent (ATCC). 4 days after the transfection, supernatant from the transfected cells was harvested and the generation of VLPs in the supernatant was confirmed.

### Example 2

### Preparation of Zika Virus Virus Like Particle

Constructs for Zika virus virus like particle prepared here are shown in FIG. 1. In the same manner as Example 1, wild type zika virus (ZIKV, Genebank# KU312312) prM-E construct (ZIKV prM-E 108F) and F108A mutant of the wild type ZIKV prM-E (ZIKV prM-E F108A, SEQ ID NOs: 5 and 6) were prepared. Chimeric zika virus prM-E with Japanese Encephalitis (JEV) envelope region was prepared (ZIKV-JEV). In detail, zika virus envelope protein stem region (404aa-504aa of SEQ ID NO: 12) was replaced with corresponding region in the JEV (nakayama strain, Genebank# EF571853) envelope region (402-502aa) by standard overwrap extension PCR method. F108A mutant of ZIKV-JEV (SEQ ID NOs: 15 and 16) was also prepared. Furthermore, a construct of furin protease recognition site-disrupted ZIKV-JEV was prepared (ZIKV-JEV-dFurin). In detail, furin recognition sequence Arg-Arg-Ser-Arg-Arg (SEQ ID NO: 19) in the zika virus prM region was altered to Factor Xa (FXa) recognition sequence Lys-Ile-Glu-Gly-Arg (SEQ ID NO: 20) by QuikChange Lightning Site-Directed Mutagenesis Kit (Agilent technologies). F108A mutant of the furin protease recognition site-disrupted ZIKV-JEV construct was also prepared (ZIKV-JEV-dFurin F108A, SEQ ID NOs: 17 and 18).

To express the VLP in mammalian cells, zika virus virus like particle expression vectors comprising the constructs shown in FIG. 1 were transfected to 293F cells using GeneX plus transfection reagent (ATCC). 4 days after the transfection, supernatant from the transfected cell was harvested and the E protein expression was examined by Western blotting using anti zika polyclonal antibody (IBT Bioservices) as a 1st antibody and rabbit anti-rabbit IgG-HRP (Santa Cruz Biotechnology) as a 2nd antibody.

As shown from FIG. 2, 293F cells transfected with the zika virus vlp expression constructs with phe at amino acid 108 position of the envelope region (108F) did not produce zika virus envelope protein in the culture supernatant. In contrast, the constructs with F108A mutation facilitated the production of envelope protein in each construct effectively.

Making a chimera of the zika virus envelope region with JEV virus envelope region and/or disrupting the furin protease recognition site in the zika prM region further enhanced the envelope protein production. Those proteins in the supernatant spontaneously assembled to give particles.

### Example 3

To confirm the particle structure, structure proteins of ZIKV-JEV-dFurin F108A were purified. The culture supernatant from construct ZIKV-JEV-dFurin F108A transfected 293F cells was harvested, then clarified by centrifugation and filtration with 0.45 μm polyethersulfone (PES) membrane. The culture supernatant was concentrated, and buffer-exchanged to 2.5 mM Sodium-Phosphate Buffer (Teknova) by KrosFlo Research II TFF Systems with mPES MidiKros Filter Modules (Spectrum Laboratories). The concentrated culture supernatant was loaded to Q XL column (GE Healthcare Life Sciences) and eluted with 2.5 to 400 mM sodium phosphate gradient. The eluates containing VLPs were concentrated by Amicon Ultra-15 centrifugal filter units (EMD Millipore) and filtered with 0.20 μm PES membrane.

Then electron microscopy analysis was performed. The purified ZIKV-JEV-dFurin F108A proteins were fixed in 4% formaldehyde and placed on glow-discharged carbon-coated 200 Mesh copper grids. The grids were then stained with 1% phosphotungstic acid and visualized by Philips CM120 Transmission Electron Microscopy at 80 kV with AMT XR80 8 megapixel camera.

About 20-30 nm diameter particle structures were detected and that indicates the presence of ZIKV-JEV-dFurin F108A as virus like particles.

### Example 4

### Preparation of a Pharmaceutical Composition Comprising Zika Virus Like Particle

Zika virus like particles were prepared according to Example 1 and Example 2. To prepare a pharmaceutical composition which is a vaccine composition, 80 μg of each of the prepared particles was mixed with 1 ml of Sucrose Phosphate Solution, pH 7.2, Endotoxin Free (Teknova, SP buffer).

What is claimed is:

1. A virus like particle comprising one or more zika virus structural proteins including envelope protein, wherein the envelope protein of the zika virus structural protein contains at least one alteration in the amino acid sequence.

2. The virus like particle according to claim 1, wherein the zika virus envelope protein has at least one alteration at a position corresponding to between amino acid position 90 and amino acid position 120 of SEQ ID NO: 11 or 12 by alignment.

3. The virus like particle according to claim 2, wherein the zika virus envelope protein has an alteration at the position corresponding to position 108 of SEQ ID NO: 11 or 12 by alignment.

4. The virus like particle according to claim 1, wherein said zika virus structural protein comprises prM and envelope proteins.

5. The virus like particle according to claim 1, wherein said zika virus structural protein has an amino acid sequence which has a sequence identity of 90% or more with any one of amino acid sequences from amino acid position 20 to the C-terminal of SEQ Nos. 1, 3, 5, 7, 15 and 17.

6. The virus like particle according to claim 5, wherein said zika virus structural protein is a protein comprising any one of amino acid sequences from amino acid position 20 to the C-terminal of SEQ Nos. 1, 3, 5, 7, 15 and 17.

7. An isolated nucleic acid molecule comprising a nucleotide sequence that encodes the zika virus structural protein contained in the virus like particle according to claim 1.

8. An isolated nucleic acid molecule consisting of a nucleotide sequence which has a sequence identity of 90% or more with a nucleotide sequence represented by any one of SEQ ID Nos: 2, 4, 6, 8, 16 and 18.

9. A vector comprising the nucleic acid molecule according to claim 7, wherein the vector optionally comprises an expression control sequence operably linked to the nucleic acid molecule.

10. A composition comprising the virus like particle according to claim 1 and a pharmaceutically acceptable carrier.

11. The composition according to claim 10 for treating or preventing a disease or condition caused by zika virus infection.

12. A DNA vaccine composition comprising the nucleic acid molecule according to claim 7.

13. The DNA vaccine composition according to claim 12 for treating or preventing a disease or condition caused by zika virus infection.

14. A method of producing the virus like particle, comprising culturing a cell which is transfected with a gene encoding the at least one zika virus structural proteins contained in the virus like particle according to claim 1; and recovering virus like particle from the cell culture.

15. A method of producing an antibody, comprising contacting the virus like particle according to claim 1 to a mammal.

16. The kit for diagnosing zika virus infection in a mammal comprising the virus like particle according to claim 1.


SEQUENCE LISTING (20 sequences)

SEQ ID NO: 1 - protein, 687 aa, zika virus
  MISC_FEATURE: Zika Virus VLP prM-E ((Strain MR766, accession number NC_012532, YP_002790881)) F108A mutant
  INIT_MET (1)..(1)
  SIGNAL (2)..(19)
  PEPTIDE (20)..(112)
  PEPTIDE (113)..(187)
  PEPTIDE (188)..(687)
  MUTAGEN (295)..(295)
MGADTSIGIIGLLLTTAMAAEITRRGSAYYMYLDRSDAGKAISFATTLGVNKCHVQIMDLGHMCDATMSYECPMLDEGVEPDDVDCWCNTTSTWVVYGTCHHKKGEARRSRRAVTLPSHSTRKLQTRSQTWLESREYTKHLIKVENWIFRNPGFALVAVAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLAGKGSLVTCAKFTCSKKMTGKSIQPENLEYRIMLSVHGSQHSGMIGYETDEDRAKVEVTPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLFSGHLKCRLKMDKLRLKGVSYSLCTAAFTFTKVPAETLHGTVTVEVQYAGTDGPCKIPVQMAVDMQTLTPVGRLITANPVITESTENSKMMLELDPPFGDSYIVIGVGDKKITHHWHRSGSTIGKAFEATVRGAKRMAVLGDTAWDFGSVGGVFNSLGKGIHQIFGAAFKSLFGGMSWFSQILIGTLLVWLGLNTKNGSISLTCLALGGVMIFLSTAVSA

SEQ ID NO: 2 - DNA, 2064 bp, zika virus
  misc_feature: Zika Virus VLP prM-E ((Strain MR766, accession number NC_012532, YP_002790881)) F108A mutant
atgggcgcagacaccagcatcggaatcattggcctcctgctgactacagccatggcagcagagatcactagacgcgggagtgcatactacatgtacttggataggagcgatgccgggaaggccatttcgtttgctaccacattgggagtgaacaagtgccacgtacagatcatggacctcgggcacatgtgtgacgccaccatgagttatgagtgccctatgctggatgagggagtggaaccagatgatgtcgattgctggtgcaacacgacatcaacttgggttgtgtacggaacctgtcatcacaaaaaaggtgaggcacggcgatctagaagagccgtgacgctcccttctcactctacaaggaagttgcaaacgcggtcgcagacctggttagaatcaagagaatacacgaagcacttgatcaaggttgaaaactggatattcaggaaccccgggtttgcgctagtggccgttgccattgcctggcttttgggaagctcgacgagccaaaaagtcatatacttggtcatgatactgctgattgccccggcatacagtatcaggtgcattggagtcagcaatagagacttcgtggagggcatgtcaggtgggacctgggttgatgttgtcttggaacatggaggctgcgttaccgtgatggcacaggacaagccaacagtcgacatagagttggtcacgacgacggttagtaacatggccgaggtaagatcctattgctacgaggcatcgatatcggacatggcttcggacagtcgttgcccaacacaaggtgaagcctaccttgacaagcaatcagacactcaatatgtctgcaaaagaacattagtggacagaggttggggaaacggttgtggacttgctggcaaagggagcttggtgacatgtgccaagtttacgtgttctaagaagatgaccgggaagagcattcaaccggaaaatctggagtatcggataatgctatcagtgcatggctcccagcatagcgggatgattggatatgaaactgacgaagatagagcgaaagtcgaggttacgcctaattcaccaagagcggaagcaaccttgggaggctttggaagcttaggacttgactgtgaaccaaggacaggccttgacttttcagacctgtattacctgaccatgaacaataagcattggttggtgcacaaagagtggtttcatgacatcccattgccttggcatgctggggcagacaccggaactccacactggaacaacaaagaggcattggtagaattcaaggatgcccacgccaagaggcaaaccgtcgtcgttctggggagccaggaaggagccgttcacacggctctcgctggagctctagaggctgagatggatggtgcaaagggaaggctgttctctggccatttgaaatgccgcctaaaaatggacaagcttagattgaagggcgtgtcatattccttgtgcactgcggcattcacattcaccaaggtcccagctgaaacactgcatggaacagtcacagtggaggtgcagtatgcagggacagatggaccctgcaagatcccagtccagatggcggtggacatgcagaccctgaccccagttggaaggctgataaccgccaaccccgtgattactgaaagcactgagaactcaaagatgatgttggagcttgacccaccatttggggattcttacattgtcataggagttggggacaagaaaatcacccaccactggcataggagtggtagcaccatcggaaaggcatttgaggccactgtgagaggcgccaagagaatggcagtcctgggggatacagcctgggacttcggatcagtcgggggtgtgttcaactcactgggtaagggcattcaccagatttttggagcagccttcaaatcactgtttggaggaatgtcctggttctcacagatcctcataggcacgctgctagtgtggttaggtttgaacacaaagaatggatctatctccctcacgtgcttggccctggggggagtgatgatcttcctctccacggctgtttctgcttaa

SEQ ID NO: 3 - protein, 684 aa, zika virus
  MISC_FEATURE: Zika Virus VLP prM-E((Strain MR766, accession number NC_012532, YP_002790881)) F108A mutant C-terminal DENV1
  INIT_MET (1)..(1)
  SIGNAL (2)..(19)
  PEPTIDE (20)..(112)
  PEPTIDE (113)..(187)
  PEPTIDE (188)..(684)
  Mutagen (295)..(295)
  MISC_FEATURE (485)..(684): DENV1 envelope protein, C-terminal region.
MGADTSIGIIGLLLTTAMAAEITRRGSAYYMYLDRSDAGKAISFATTLGVNKCHVQIMDLGHMCDATMSYECPMLDEGVEPDDVDCWCNTTSTWVVYGTCHHKKGEARRSRRAVTLPSHSTRKLQTRSQTWLESREYTKHLIKVENWIFRNPGFALVAVAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLAGKGSLVTCAKFTCSKKMTGKSIQPENLEYRIMLSVHGSQHSGMIGYETDEDRAKVEVTPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLFSGHLKCRLKMDKLRLKGMSYVMCTGSFKLEKEVAETQHGTVLVQVKYEGTDAPCKIPFSSQDEKGVTQNGRLITANPIVTDKEKPVNIEAEPPFGESYIVVGAGEKALKLSWFKKGSSIGKMFEATARGARRMAILGDTAWDFGSIGGVFTSVGKLIHQIFGTAYGVLFSGVSWTMKIGIGILLTWLGLNSRSTSLSMTCIAVGMVTLYLGVMVQA

SEQ ID NO: 4 - DNA, 2055 bp, zika Virus
  misc_feature: Zika Virus VLP prM-E((Strain MR766, accession number NC_012532, YP_002790881)) F108A mutant C-terminal DENV1
atgggcgcagacaccagcatcggaatcattggcctcctgctgactacagccatggcagcagagatcactagacgcgggagtgcatactacatgtacttggataggagcgatgccgggaaggccatttcgtttgctaccacattgggagtgaacaagtgccacgtacagatcatggacctcgggcacatgtgtgacgccaccatgagttatgagtgccctatgctggatgagggagtggaaccagatgatgtcgattgctggtgcaacacgacatcaacttgggttgtgtacggaacctgtcatcacaaaaaaggtgaggcacggcgatctagaagagccgtgacgctcccttctcactctacaaggaagttgcaaacgcggtcgcagacctggttagaatcaagagaatacacgaagcacttgatcaaggttgaaaactggatattcaggaaccccgggtttgcgctagtggccgttgccattgcctggcttttgggaagctcgacgagccaaaaagtcatatacttggtcatgatactgctgattgccccggcatacagtatcaggtgcattggagtcagcaatagagacttcgtggagggcatgtcaggtgggacctgggttgatgttgtcttggaacatggaggctgcgttaccgtgatggcacaggacaagccaacagtcgacatagagttggtcacgacgacggttagtaacatggccgaggtaagatcctattgctacgaggcatcgatatcggacatggcttcggacagtcgttgcccaacacaaggtgaagcctaccttgacaagcaatcagacactcaatatgtctgcaaaagaacattagtggacagaggttggggaaacggttgtggacttgctggcaaagggagcttggtgacatgtgccaagtttacgtgttctaagaagatgaccgggaagagcattcaaccggaaaatctggagtatcggataatgctatcagtgcatggctcccagcatagcgggatgattggatatgaaactgacgaagatagagcgaaagtcgaggttacgcctaattcaccaagagcggaagcaaccttgggaggctttggaagcttaggacttgactgtgaaccaaggacaggccttgacttttcagacctgtattacctgaccatgaacaataagcattggttggtgcacaaagagtggtttcatgacatcccattgccttggcatgctggggcagacaccggaactccacactggaacaacaaagaggcattggtagaattcaaggatgcccacgccaagaggcaaaccgtcgtcgttctggggagccaggaaggagccgttcacacggctctcgctggagctctagaggctgagatggatggtgcaaagggaaggctgttctctggccatttgaaatgccgcctaaaaatggacaagcttagattgaagggcatgtcatatgtaatgtgcacagggtcattcaagttagagaaggaagtggctgagacccagcatggaactgttctagtgcaggttaaatacgaaggaacagatgcaccatgcaagatccccttctcgtcccaagatgagaagggagtaacccagaatgggagattgataacagccaaccccatagtcactgacaaagaaaaaccagtcaacattgaagcggagccaccttttggtgagagctacattgtggtaggagcaggtgaaaaagctttgaaactaagctggttcaagaagggaagcagtatagggaaaatgtttgaagcaactgcccgtggagcacgaaggatggccatcctgggagacactgcatgggacttcggttctataggaggggtgttcacgtctgtgggaaaactgatacaccagatttttgggactgcgtatggagttttgttcagcggtgtttcttggaccatgaagataggaatagggattctgctgacatggctaggattaaactcaaggagcacgtccctttcaatgacgtgtatcgcagttggcatggtcacgctgtacctaggagtcatggttcaggcgtaa

SEQ ID NO: 5 - protein, 691 aa, zika virus
  MISC_FEATURE: Zika virus VLP prM-E (Strain Z1106033, accession number KU312312) F108A mutant
  INIT_MET (1)..(1)
  SIGNAL (2)..(19)
  PEPTIDE (20)..(112)
  PEPTIDE (113)..(187)
  PEPTIDE (188)..(691)
  MUTAGEN (295)..(295)
MGADTSVGIVGLLLTTAMAAEVTRRGSAYYMYLDRNDAGEAISFPTTLGMNKCYIQIMDLGHTCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLAGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNAKNGSISLMCLALGGVLIFLSTAVSA

SEQ ID NO: 6 - DNA, 2076 bp, zika virus
  misc_feature: Zika virus VLP prM-E (Strain Z1106033, accession number KU312312) F108A mutant
atgggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacacgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggaggtctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgtcactgtaatggcacaggacaaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggacttgctggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaagttgagataacgcccaattcaccaagagccgaagccaccctgggggggtttggaagcctaggacttgattgtgaaccgaggacaggccttgacttttcagatttgtattacttgactatgaataacaagcactggctggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtactgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatctttggagcagctttcaaatcattgtttggaggaatgtcctggttctcacaaattctcattggaacgttgctgatgtggttgggtctgaacgcaaagaatggatctatttcccttatgtgcttggccttagggggagtgttgatcttcttatccacagccgtctctgcttaa

SEQ ID NO: 7 - protein, 688 aa, zika virus
  MISC_FEATURE: Zika virus VLP prM-E (Strain Z1106033, accession number KU312312) F108A mutant C-terminal DENV1
  INIT_MET (1)..(1)
  SIGNAL (2)..(19)
  PEPTIDE (20)..(112)
  PEPTIDE (113)..(187)
  PEPTIDE (188)..(688)
  MISC_FEATURE (490)..(688): DENV1 envelope protein, C-terminal region.
MGADTSVGIVGLLLTTAMAAEVTRRGSAYYMYLDRNDAGEAISFPTTLGMNKCYIQIMDLGHTCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLAGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGMSYVMCTGSFKLEKEVAETQHGTVLVQVKYEGTDAPCKIPFSSQDEKGVTQNGRLITANPIVTDKEKPVNIEAEPPFGESYIVVGAGEKALKLSWFKKGSSIGKMFEATARGARRMAILGDTAWDFGSIGGVFTSVGKLIHQIFGTAYGVLFSGVSWTMKIGIGILLTWLGLNSRSTSLSMTCIAVGMVTLYLGVMVQA

SEQ ID NO: 8 - DNA, 2064 bp, zika virus
  misc_feature: Zika virus VLP prM-E (Strain Z1106033, accession number KU312312) F108A mutant C-terminal DENV1
atgggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacacgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggaggtctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgtcactgtaatggcacaggacaaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggacttgctggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaagttgagataacgcccaattcaccaagagccgaagccaccctgggggggtttggaagcctaggacttgattgtgaaccgaggacaggccttgacttttcagatttgtattacttgactatgaataacaagcactggctggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcatgtcatatgtaatgtgcacagggtcattcaagttagagaaggaagtggctgagacccagcatggaactgttctagtgcaggttaaatacgaaggaacagatgcaccatgcaagatccccttctcgtcccaagatgagaagggagtaacccagaatgggagattgataacagccaaccccatagtcactgacaaagaaaaaccagtcaacattgaagcggagccaccttttggtgagagctacattgtggtaggagcaggtgaaaaagctttgaaactaagctggttcaagaagggaagcagtatagggaaaatgtttgaagcaactgcccgtggagcacgaaggatggccatcctgggagacactgcatgggacttcggttctataggaggggtgttcacgtctgtgggaaaactgatacaccagatttttgggactgcgtatggagttttgttcagcggtgtttcttggaccatgaagataggaatagggattctgctgacatggctaggattaaactcaaggagcacgtccctttcaatgacgtgtatcgcagttggcatggtcacgctgtacctaggagtcatggttcaggcg

SEQ ID NO: 9 - protein, 19 aa, zika virus
  MISC_FEATURE: Strain MR766, accession number NC_012532, YP_002790881, Initiation codon M and signal peptide
  INIT_MET (1)..(1)
  SIGNAL (2)..(19)
MGADTSIGIIGLLLTTAMA

SEQ ID NO: 10 - protein, 19 aa, zika virus
  MISC_FEATURE: Strain Z1106033, accession number KU312312, Initiation codon M and signal peptide
  INIT_MET (1)..(1)
  SIGNAL (2)..(19)
MGADTSVGIVGLLLTTAMA

SEQ ID NO: 11 - protein, 500 aa, zika virus
  MISC_FEATURE: Strain MR766, accession number NC_012532, YP_002790881, Envelope protein
  PEPTIDE (1)..(500)
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFTCSKKMTGKSIQPENLEYRIMLSVHGSQHSGMIGYETDEDRAKVEVTPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLFSGHLKCRLKMDKLRLKGVSYSLCTAAFTFTKVPAETLHGTVTVEVQYAGTDGPCKIPVQMAVDMQTLTPVGRLITANPVITESTENSKMMLELDPPFGDSYIVIGVGDKKITHHWHRSGSTIGKAFEATVRGAKRMAVLGDTAWDFGSVGGVFNSLGKGIHQIFGAAFKSLFGGMSWFSQILIGTLLVWLGLNTKNGSISLTCLALGGVMIFLSTAVSA

SEQ ID NO: 12 - protein, 504 aa, zika virus
  MISC_FEATURE: Strain Z1106033, accession number KU312312, Envelope protein
  PEPTIDE (1)..(504)
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNAKNGSISLMCLALGGVLIFLSTAVSA

SEQ ID NO: 13 - protein, 497 aa, dengue virus
  MISC_FEATURE: Dengue type 1 virus, Western Pacific strain, Genbank# U88535.1, envelope protein
  PEPTIDE (1)..(497)
MAMRCVGIGNRDFVEGLSGATWVDVVLEHGSCVTTMAKDKPTLDIELLKTEVTNPAVLRKLCIEAKISNTTTDSRCPTQGEATLVEEQDTNFVCRRTFVDRGWGNGCGLFGKGSLITCAKFKCVTKLEGKIVQYENLKYSVIVTVHTGDQHQVGNETTEHGTTATITPQAPTSEIQLTDYGALTLDCSPRTGLDFNEMVLLTMEKKSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQVKYEGTDAPCKIPFSSQDEKGVTQNGRLITANPIVTDKEKPVNIEAEPPFGESYIVVGAGEKALKLSWFKKGSSIGKMFEATARGARRMAILGDTAWDFGSIGGVFTSVGKLIHQIFGTAYGVLFSGVSWTMKIGIGILLTWLGLNSRSTSLSMTCIAVGMVTLYLGVMVQA

SEQ ID NO: 14 - protein, 500 aa, Japanese encephalitis virus
  MISC_FEATURE: Japanese Encephalitis virus, nakayama strain, Genebank# EF571853, envelope protein.
  PEPTIDE (1)..(500)
  misc_feature (209)..(209): Xaa can be any naturally occurring amino acid
FNCLGMGNRDFIEGASGATWVDLVLEGDSCLTIMANDKPTLDVRMINIEAVQLAEVRSYCYHASVTDISTVARCPTTGEAHNKKRADSSYVCKQGFTDRGWGNGCGLFGKGSIDTCAKFSCTSKAIGRTIQPENIKYEVGIFVHGTTTSENHGNYSAQVGASQAAKFTVTPNAPSTTLKLGDYGEVTLDCEPRSGLNTEAFYVMTVGSXSFLVHREWFHDLALPWTPPSSTAWRNRELLMEFEEAHATKQSVVALGSQEGGLHQALAGAIVVEYSNSVKLTSGHLKCRLRMDKLALKGTTYGMCTEKFSFAKNPADTGHGTVVIELSYSGSDGPCKIPIVSVASLNDMTPVGRLVTVNPFVATSSANSKVLVEMEPPFGDSYIVVGRGDKQINHHWHKAGSTLGKAFSTTLKGAQRLAALGDTAWDFGSIGGVFNSIGKAVHQVFGGAFRTLFGGMSWITQGLMGALLLWMGVNARDRSIALAFLATGGVLVFLATNVHA

SEQ ID NO: 15 - protein, 691 aa, zika virus
  MISC_FEATURE: Zika virus VLP prM-E (Strain Z1106033, accession number KU312312) F108A mutant C-terminal JEV
  INIT_MET (1)..(1)
  SIGNAL (2)..(19)
  MUTAGEN (295)..(295)
  MISC_FEATURE (591)..(691): Janapese Encephalitis Virus Envelope protein, C-terminal region.
MGADTSVGIVGLLLTTAMAAEVTRRGSAYYMYLDRNDAGEAISFPTTLGMNKCYIQIMDLGHTCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLAGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTLGKAFSTTLKGAQRLAALGDTAWDFGSIGGVFNSIGKAVHQVFGGAFRTLFGGMSWITQGLMGALLLWMGVNARDRSIALAFLATGGVLVFLATNVHA

SEQ ID NO: 16 - DNA, 2076 bp, zIka virus
  misc_feature: Zika virus VLP prM-E (Strain Z1106033, accession number KU312312) F108A mutant C-terminal JEV
  mutation (883)..(884)
  misc_feature (1771)..(2076): Japanese encephalitis virus envelope protein, C-terminal region.
atgggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacacgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggaggtctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgtcactgtaatggcacaggacaaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggacttgctggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaagttgagataacgcccaattcaccaagagccgaagccaccctgggggggtttggaagcctaggacttgattgtgaaccgaggacaggccttgacttttcagatttgtattacttgactatgaataacaagcactggctggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtactgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggaagcacgctgggcaaagccttttcaacgactttgaagggagctcaaagactggcagcgttgggcgacacagcctgggactttggctctattggaggggttttcaactccatagggaaagccgttcaccaagtgtttggtggtgccttcagaacactcttcgggggaatgtcttggatcacacaagggctaatgggggccctactactctggatgggcgtcaacgcacgagaccgatcaattgctctggccttcttagccacaggaggtgtgctcgtgttcttagcgaccaatgtgcatgcttga

SEQ ID NO: 17 - protein, 691 aa, zika virus
  MISC_FEATURE: Furin protease recognition site-disrupted zika virus VLP prM-E (Strain Z1106033, accession number KU312312) F108A mutant C-terminal Japanese encephalitis virus.
  INIT_MET (1)..(1)
  SIGNAL (2)..(19)
  MUTAGEN (108)..(112): Factor Xa recognition Sequence.
  MUTAGEN (295)..(295)
  MISC_FEATURE (591)..(691): Japanese encephalitis virus envelope protein, C-terminal region.
MGADTSVGIVGLLLTTAMAAEVTRRGSAYYMYLDRNDAGEAISFPTTLGMNKCYIQIMDLGHTCDATMSYECPMLDEGVEPDDVDCWCNTTSTWVVYGTCHHKKGEAKIEGRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLAGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTLGKAFSTTLKGAQRLAALGDTAWDFGSIGGVFNSIGKAVHQVFGGAFRTLFGGMSWITQGLMGALLLWMGVNARDRSIALAFLATGGVLVFLATNVHA

SEQ ID NO: 18 - DNA, 2076 bp, zika virus
  misc_feature: Furin protease recognition site-disrupted zika virus VLP prM-E (Strain Z1106033, accession number KU312312) F108A mutant C-terminal Japanese encephalitis virus.
  mutation (318)..(318)
  mutation (322)..(336)
  mutation (339)..(339)
atgggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacacgtgtgatgccaccatgagctatgaatgccctatgctggatgagggggtggaaccagatgacgtcgattgttggtgcaacacgacgtcaacttgggttgtgtacggaacctgtcatcacaaaaaaggtgaggcaaagatagagggaagagccgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgtcactgtaatggcacaggacaaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggacttgctggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaagttgagataacgcccaattcaccaagagccgaagccaccctgggggggtttggaagcctaggacttgattgtgaaccgaggacaggccttgacttttcagatttgtattacttgactatgaataacaagcactggctggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtactgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggaagcacgctgggcaaagccttttcaacgactttgaagggagctcaaagactggcagcgttgggcgacacagcctgggactttggctctattggaggggttttcaactccatagggaaagccgttcaccaagtgtttggtggtgccttcagaacactcttcgggggaatgtcttggatcacacaagggctaatgggggccctactactctggatgggcgtcaacgcacgagaccgatcaattgctctggccttcttagccacaggaggtgtgctcgtgttcttagcgaccaatgtgcatgcttga

SEQ ID NO: 19 - protein, 5 aa, Artificial Sequence
  Synthetic peptide sequence
RRSRR

SEQ ID NO: 20 - protein, 5 aa, Artificial Sequence
  Synthetic peptide sequence
KIEGR